(19) United States
(12) Patent Application Publication
(10) Pub. No.: US 2006/0269580 A1
(43) Pub. Date: Nov. 30, 2006

(54) Treatment of skin with light and a benefit agent to mitigate acne

(76) Inventors: Curtis A. Cole, Ringoes, NJ (US); Jean Lukenbach, Flemington, NJ (US); Gregory Skover, Princeton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/183,420

(22) Filed: Jul. 15, 2005

Related U.S. Application Data
(60) Provisional application No. 60/588,659, filed on Jul. 16, 2004.

(51) Int. Cl. A61K 8/64; A61K 8/02; A61H 21/00

(52) U.S. Cl. 424/401; 607/86; 514/2

(57) ABSTRACT

Methods of mitigating acne include exposing an expanse of skin to light; terminating the exposure of the skin to the light; and applying a benefit agent to the expanse of skin after a delay following the termination. The light exposure may be for a period of less than about one hour, and the light may be suitable for either (a) exciting porphyrins associated with the expanse of skin into an energetic state suitable for destroying acne-causing micro-organisms, or (b) for heating lipids present in sebaceous glands within the expanse of skin in order to modulate the flow of sebum in said sebaceous glands, or (c) for reducing inflammation. The benefit agent is suitable for either (a) providing anti-microbial action that is complementary to either said modulating of said sebum by said band of light, or complementary to said reduction of inflammation by said light or (b) providing sebum-modulating action that is complementary to either said destruction of said acne-causing microorganisms or complementary to said reduction of inflammation by said light; or (c) providing anti-inflammation that is complementary to either said modulating of said sebum by said band of light, or complementary to said destruction of said acne-causing microorganisms. These treatments may be repeated, as necessary.

### FIELD OF THE INVENTION

The present invention relates to treatment of the skin and, more particularly, to the application of light to the skin, followed by the topical application of a benefit agent to said skin.

### BACKGROUND OF THE INVENTION

Acne and rosacea are major diseases of the skin associated with sebaceous follicles on the skin. There are many treatments, but no cures for these diseases. Such treatments for acne include antimicrobials such as benzoyl peroxide which kill or inhibit growth of p. acnes bacteria which play a role in acne; sebum modulating agents such as retinoids, including tretinoin and isotetinoin which influence sebum production; keratolytic agents such as salicylic acid which accelerate cell turnover and open hair follicles; anti-inflammatories such as dimethyl aminoethanol (DMAE) to reduce redness and pain associated with acne lesions; cleansing agents such as alcohols to open the infindibulum and allow free sebum exit to the skin surface; anti-spot/pigmentation agents such as ascorbic acid to prevent or treat pigmentation and color contrast on the skin; and anti-scar agents such as copper peptides to reduce the impact of scar formation from acne lesions. Rosacea can be treated with antibiotics, sulfur, sodium sulfacetamide, and retinoids.

It has also been proposed to treat acne by exposing the skin to electromagnetic radiation. The electromagnetic radiation typically includes wavelengths that are suitable to photochemically activate compounds such as endogenous porphyrins or their biochemical building blocks that are topically applied to the skin.

For example, McDaniel (U.S20030004499 and WO2003001894) teaches a method for dermatological treatment using narrowband, multichromatic electromagnetic radiation. A topical pre-treatment, such as an exogenous chromophore or a cosmaeceutical may be used to enhance the penetration of light. The procedure may be repeated every 1 to 60 days.

Korman (US20020128695A1) teaches a method for high-energy photodynamic therapy of acne vulgaris and seborrhea. The method includes illuminating a skin area with narrow-band, high intensity light having spectral characteristics of at least one of a group of narrow spectral bands consisting of 400 nm-450 nm (blue), 520 nm-550 nm (green) or 630 nm-670 nm (red) spectral range. The light source generates a high intensity, non-coherent light in exact narrow spectral bands needed for activation of the photodynamic reaction while filtering out harmful UV light. Pre-treatment with oxygen transporting compounds, perfluorocarbons, oxidative substances such as a hydrogen peroxide compound, keratolytic substances and external photosensitizers such as Methylene blue may be performed. The function of these pre-treatments is to release oxygen directly into the sebaceous glands and raise the efficiency of the destruction of p. acnes.

Perricone (US20030009158A1) describes using topical treatments such as glycolic acid to enhance penetration of light or block light below the desired wavelengths (between 400 nm-590 nm). Such treatments may be applied to prior to or during phototreatrnent to increase light penetration into the skin. Chemical filters to remove light that is not within this desired range. Fat-soluble fatty acid esters of ascorbic acid may be applied to the skin before, during, or after blue/violet light treatments.

Anderson (US20020099094) teaches light treatment of sebaceous gland disorders with 5-aminolevulinic acid (ALA) and photodynamic therapy. The ALA is described as metabolized via the porphyrin pathway. A metabolite infiltrates the skin to be treated. When intense light with a wavelength between 320 and 700 is delivered to ALA-treated skin, the metabolite metabolite (photoporphyrin IX) is excited and reacts with oxygen to produce singlet oxygen, modulating sebaceous gland disorders such as acne.

Anderson (U.S. Pat. No. 6,183,773) describes a method of treating a sebaceous gland disorder by topically applying a chromophore or an “energy-activatable material” such as methylene blue, causing it to infiltrate into spaces of the skin, and exposing the skin to energy to photochemically activate the chromophore. The chromophore should have an absorption spectrum in the range of 600 nm to 1300 nm to minimize surrounding blood from absorbing from absorbing light intended for the chromophore.

The preceding examples illustrate that conventional treatment of the skin using electromagnetic radiation employs a monotherapy approach. For example, in conventional treatment, the skin is exposed to electromagnetic radiation, perhaps after a chromophore or a porphyrin precursor is topically applied thereto. The skin and the chromophore or a porphyrin precursor absorb radiation to raise the efficiency of the destruction of p. acnes. As such, only a single biological pathway (thermal injury/recovery) is employed to affect a particular benefit. This is unfortunate, since this solitary mechanism is prone to diminishing returns as the fluence, frequency or time of radiation is increased. In many cases, saturation of the benefit is achieved beyond a certain frequency, fluence, or time of treatment.

Accordingly, conventional practices are subject to several drawbacks. Firstly, electromagnetic radiation having a high energy density (fluence) is often utilized. The high energy density delivered may be unsafe for a lay user (e.g., a consumer) to use in a home setting. Furthermore, high fluence radiation tends to heat the skin to an uncomfortable temperature and therefore require that the skin be cooled during operation. For example, for devices that contact the skin, this uncomfortable heating may require that a skin-cooling system be built into the device itself, which can be expensive or limiting to the device design.

For other conventional practices, the fluence of radiation is too low to deliver adequate efficacy. Even if the patient goes through the inconvenience and expense of making frequent visits to a professional skin care specialist to receives multiple treatments, the results are often unsatisfactory. Furthermore, treatment with electromagnetic radiation alone does not impart protection from further aging-related degradation of the treated tissue that may result in the future.

Therefore, a need exists for a system for treating the skin that overcomes one or more of the above-mentioned drawbacks.

### SUMMARY OF THE INVENTION

In one aspect, embodiments of the invention relate to a method of mitigation of acne. In a first embodiment, the method includes exposing an expanse of skin to light; terminating the exposure of the skin to the light; and applying a benefit agent to the expanse of skin after a delay following the termination. The light exposure may be for a period of less than about one hour, and the light may be suitable for either (a) exciting porphyrins associated with the expanse of skin into an energetic state suitable for destroying acne-causing micro-organisms, or (b) for heating lipids present in sebaceous glands within the expanse of skin in order to modulate the flow of sebum in said sebaceous glands, or (c) for reducing inflammation. The benefit agent is suitable for either (a) providing anti-microbial action that is complementary to either said modulating of said sebum by said band of light, or complementary to said reduction of inflammation by said light or (b) providing sebum-modulating action that is complementary to either said destruction of said acne-causing microorganisms or complementary to said reduction of inflammation by said light; or (c) providing anti-inflammation that is complementary to either said modulating of said sebum by said band of light, or complementary to said destruction of said acne-causing microorganisms.

In another embodiment, the method includes providing a first skin treatment to an expanse of skin, and after a delay, providing a second skin treatment to the same expanse of skin. The first skin treatment includes initiating exposure of an expanse of skin to light; terminating the exposure of the expanse of skin to the light after a period, preferably of less than about one hour; and applying a first benefit agent treatment to the expanse of skin after a first delay following the termination. The light is primarily within about 400 nm to about 850 nm with a fluence of about 5 J/cm2 to about 100 J/cm2, and it may be suitable for either (a) exciting porphyrins associated with the expanse of skin into an energetic state suitable for destroying acne-causing micro-organisms, or (b) for heating lipids present in sebaceous glands within the expanse of skin in order to modulate the flow of sebum in said sebaceous glands, or (c) for reducing inflammation. The benefit agent may be suitable for either (a) providing anti-microbial action that is complementary to either said modulating of said sebum by said band of light, or complementary to said reduction of inflammation by said light or (b) providing sebum-modulating action that is complementary to either said destruction of said acne-causing microorganisms or complementary to said reduction of inflammation by said light; or (c) providing anti-inflammation that is complementary to either said modulating of said sebum by said band of light, or complementary to said destruction of said acne-causing microorganisms. The second skin treatment includes initiating exposure of an expanse of skin to light; terminating the exposure of the expanse of skin to the light after a period, preferably of less than about one hour; and applying a the first benefit agent treatment to the expanse of skin after a delay following the termination. This delay may be similar to the first delay, but the second delay is preferably of greater duration that the first delay. At least one additional benefit agent treatment may also be applied during the second delay.

In another aspect of the invention, a method of promoting a topical composition, the method includes the steps of instructing a user to topically apply said composition to an expanse of skin following an exposure of said expanse of skin to light. The light is substantially free of ultraviolet radiation; is primarily within about 400 nm to about 850 nm; and provides a fluence of about 5 J/cm2 to about 100 J/cm2, having selected wavelengths and/or wavelength bands, primarily within the spectral range of about 400 nm to about 850 nm wherein said light source delivered from about 0.01 Watt/cm2 to about 100 W/cm2 to the skin wherein the total fluence delivered is less than 100 J/cm2. Preferably, the light exposure is completed within 24 hours prior to said topical application. The benefit agent may be suitable for either (a) providing anti-microbial action that is complementary to either said modulating of said sebum by said band of light, or complementary to said reduction of inflammation by said light or (b) providing sebum-modulating action that is complementary to either said destruction of said acne-causing microorganisms or complementary to said reduction of inflammation by said light; or (c) providing anti-inflammation that is complementary to either said modulating of said sebum by said band of light, or complementary to said destruction of said acne-causing microorganisms.

In another aspect of the invention, a kit includes a light source, a benefit agent, and instructions. The light source provides a fluence of about 5 J/cm² to about 100 J/cm² of light primarily within about 400 nm to about 800 nm, and/or the light and it is suitable for either (a) exciting porphyrins associated with the expanse of skin into an energetic state suitable for destroying acne-causing micro-organisms, or (b) for heating lipids present in sebaceous glands within the expanse of skin in order to modulate the flow of sebum in said sebaceous glands, or (c) for reducing inflammation. The benefit agent may be suitable for either (a) providing anti-microbial action that is complementary to either said modulating of said sebum by said band of light, or complementary to said reduction of inflammation by said light or (b) providing sebum-modulating action that is complementary to either said destruction of said acne-causing microorganisms or complementary to said reduction of inflammation by said light; or (c) providing anti-inflammation that is complementary to either said modulating of said sebum by said band of light, or complementary to said destruction of said acne-causing microorganisms. The instructions relate to the application of at least one treatment of the benefit agent to the skin within 24 hours immediately following exposure of skin to light from said light source.

### BRIEF DESCRIPTION OF THE DRAWINGS

A more particular description of the invention, briefly summarized above may be had by reference to the embodiments thereof that are illustrated in the appended drawings. It is to be so noted, however, that the appended drawings illustrate only typical embodiments of the invention and, therefore, are not to be considered limiting of its scope, for the invention may admit to other equally effective embodiments.

FIG. 1 is a schematic side view of an expanse of skin being treated with light, according to embodiments of the invention described herein;

FIG. 2a is a schematic top view of an expanse of skin being treated with light;

FIG. 2b is a schematic top view of an expanse of skin, and light being progressively repositioned across the expanse of skin; and

FIG. 3 is a schematic side view of a device capable of being progressively repositioned across an expanse of skin in a manner consistent with embodiments of the invention described herein.

To facilitate understanding identical reference elements have been used, wherever possible, to designate identical elements that are common to the figures.

### DETAILED DESCRIPTION OF THE INVENTION

Unless defined otherwise, all technical and scientific terms used herein have the same meaning as commonly understood by one of ordinary skill in the art to which the invention belongs.

Embodiments of the invention includes apparatus and methods for mitigating acne. By “mitigating acne,” it is meant one or more of the following benefits are imparted to a subject's skin: reduction in the number, size, volume, color contrast, tactile pain, and/or obtrusiveness of acne lesions, rosacea, and the potential for causing long-term marks or scars of the skin surface.

Light Treatment

In order mitigate acne at least one skin treatment is provided. By providing a “skin treatment,” it is meant initiating exposure of an expanse of skin to light, terminating exposure to the light; and applying at least one benefit agent to the expanse of skin after a delay following the termination of the exposure to the light. Thus a “skin treatment” includes a light treatment followed by a topical treatment.

The skin treatment may be directed to a large area (e.g., an entire face). For example, an entire face of a subject may be simultaneously exposed to light, after which a topical benefit agent is applied to the entire face. This type of treatment may be suitable for preventing or treating acne lesions, rosacea, scarring, and the like that spread across an area larger than a few square centimeters. Alternatively, the skin treatment may be directed to a small area (e.g., for “spot treating” an emerging individual acne lesion). For example, in this embodiment of the invention, light may be directed to one individual lesion at a time without necessarily exposing the entire face. After this light treatment, a topical benefit agent is then applied to the lesion. This type of treatment may be suitable for treating acute acne lesions, rosacea, scarring, and the like that is localized to an area that is, for example, less than 10 square centimeters. More detail regarding exposing the skin to light and administering a benefit agent is provided below.

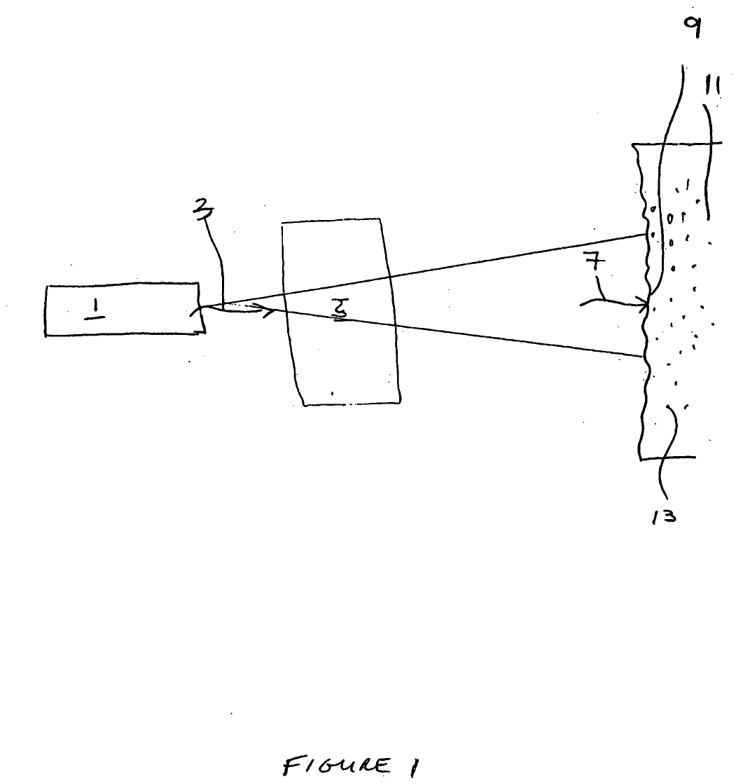

Referring to FIG. 1, a light source 1 is used to provide light treatment. Generally, the light source 1 is a pulsed or continuous wave source that emits an emitted light 3. The emitted light 3 may be spectrally concentrated or spectrally diffuse (i.e., broadband). The emitted light 3 may be subsequently filtered, attenuated, amplified, polarized, or otherwise modified by one or more optical elements 5 before it reaches an expanse of skin 11 to which it is directed. At the point which the light reaches an outer surface 9 of the expanse of skin 11 interacts with the skin, the light consists of an incident light 7.

The incident light 7 comprises an “active portion” that is specifically target towards mitigating acne via one of three particular pathways. For example, in one embodiment of the invention, the incident light 7 is specifically targeted towards (1) antimicrobial action, i.e., the destruction of microorganisms such as acne-causing bacteria. As such, the incident light 7 may include wavelengths primarily within a spectral range suitable for activating endogenous porphyrins or metabolites thereof to enact the photodestruction of p. acnes. For example, the incident light 7 may be primarily within a range of wavelengths defined by the union of (a) wavelengths between 400 nm and 450 nm (such as between about 400 nm and about 410 nm); and (b) wavelengths between about 600 nm and about 700 nm (such as between about 630 nm and about 670 nm).

Alternatively, in another embodiment of the invention, the incident light 7 is specifically targeted towards (2) sebum modulation or sebum heating. As such, the incident light 7 may primarily within a range of wavelengths defined by the union of (c) wavelengths between about 700 and 2000 nm (such as between 1000 nm and 1800 nm); and (d) wavelengths between about 575 nm and 625nm.

Alternatively, in another embodiment of the invention, the incident light 7 is specifically targeted towards (3) inflammation control to provide one or more of the following benefits: minimization of pain, redness and post-lesion pigmentation and scarring. As such, the incident light 7 is primarily within a range of wavelengths (e) between about 600 nm and about 750 nm (such as between about 600 nm and about 700 nm).

Note that in each of the three embodiments of the invention described above, the emitted light and/or the incident light may or may not also include wavelengths outside of the particular active portion discussed above, but emission outside of the particular active portion is not required.

In one embodiment of the invention, the incident light is primarily within one of the spectral ranges identified above. By “primarily within” it is meant that 80% or more of the total energy of the incident light is within the identified spectral range. In one embodiment of the invention, the incident light 7 is substantially within the identified spectral range. By “substantially within the spectral range” it is meant that 90% or more of the total energy is within the identified spectral range. In another embodiment of the invention, in order to limit damage to the skin from ultraviolet radiation, the incident light 7 is substantially free of ultraviolet radiation (i.e., less than about 1% of the total energy of the incident light 7 is in the spectral range from about 200 nm to about 400 nm).

The active portion is generally capable of being absorbed by one or more types of endogenous chromophores 13 present within the expanse of skin 11. The chromophores 13 include one or more of the following compounds: melanin, hemoglobin, deoxyhemoglobin, sebum and water.

In one embodiment of the invention, the incident light 7 is not energetic enough to ablate the epidermis. As such the incident light 7 impinges upon the expanse of skin 11 with an energy density that is generally sufficient to provide localized thermal heating (such as to, for example, raise the temperature of the skin by less than about 10 Celsius degrees) and a beneficial wound-healing response.

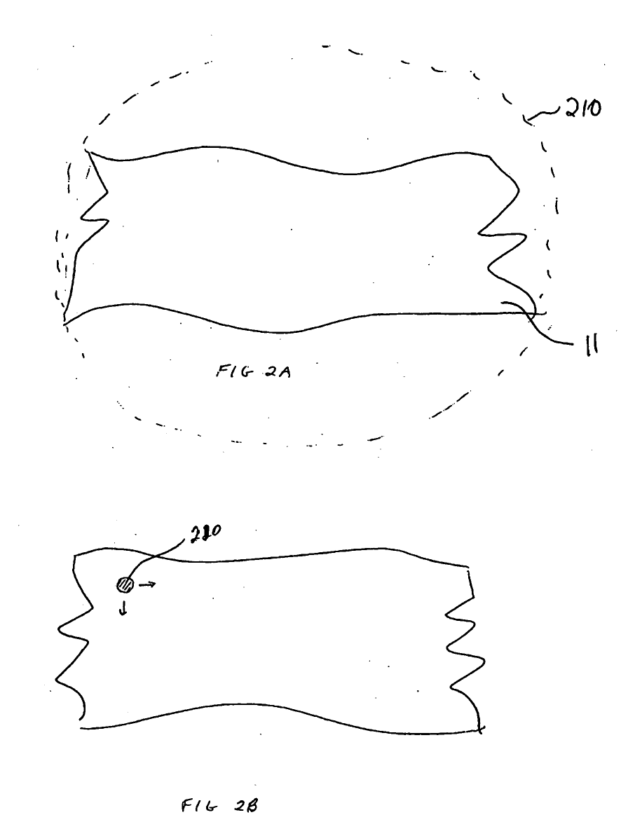

The energy density of the incident light 7 may be within a range of about 5 J/cm² and about 100 J/cm², such as between about 5 J/cm² and about 50 J/cm2. By “energy density of the incident light” 7, it is meant the energy of the incident light 7 divided by the area of a spot 210, as shown in FIG. 2A, over which the energy extends, the area determined as it impinges upon the outer surface 9 of the expanse of skin 11. Note that the terms “energy density” and “fluence” are used interchangeably throughout this disclosure. The spot 210 may have an area of about 0.5 cm² to about 10 cm². Area of spot 210 is also referred to as “spot size” in this specification.

The energy density of the incident light 7 may be delivered over a particular time that may be, for example in a range of about 1 millisecond (msec) to about 60 minutes. Note that shorter times are generally more suitable for higher fluence, and longer times are more suitable for lower fluence.

The incident light 7 or the active portion thereof may impinge upon the expanse of skin 11 with an irradiance that is in a range from about 1 milliwatt per square centimeter (mW/cm²) to about 100,000 watts per square centimeter (W/cm²). “Irradiance” of the incident light, is the energy density of the incident light 7 delivered to the expanse of skin 11 per unit time period.

The spot 210 may, in one embodiment of the invention, as shown in FIG. 2A, fully encompass the expanse of skin 11 to be treated. In this embodiment, there is no need for the incident light to be progressively repositioned (e.g., moved laterally across the expanse of skin 11) in order to deliver energy to the expanse of skin 11 across its entirety. Alternatively, as shown in FIG. 2B, the incident light 7 may have a spot 210 that is relatively small in area, e.g., less than about 1 cm², and may be progressively repositioned (e.g., stamped) across the expanse of skin 11 in order to treat the entire expanse of skin 11.

The incident light 7, or active portion thereof has a bandwidth. The bandwidth is determined by finding a wavelength (i.e., a maxima) within the active portion that is of maximum intensity, dividing this intensity in half (a “half max”) and locating a nearest first wavelength in one spectral direction that is incident at that half max intensity. A nearest second wavelength in the other spectral direction is then located. The difference between the first wavelength and the second wavelength is calculated as the bandwidth. Note that if multiple maxima are incident on the expanse of skin 11, then the maxima of greatest intensity is chosen to calculate the bandwidth.

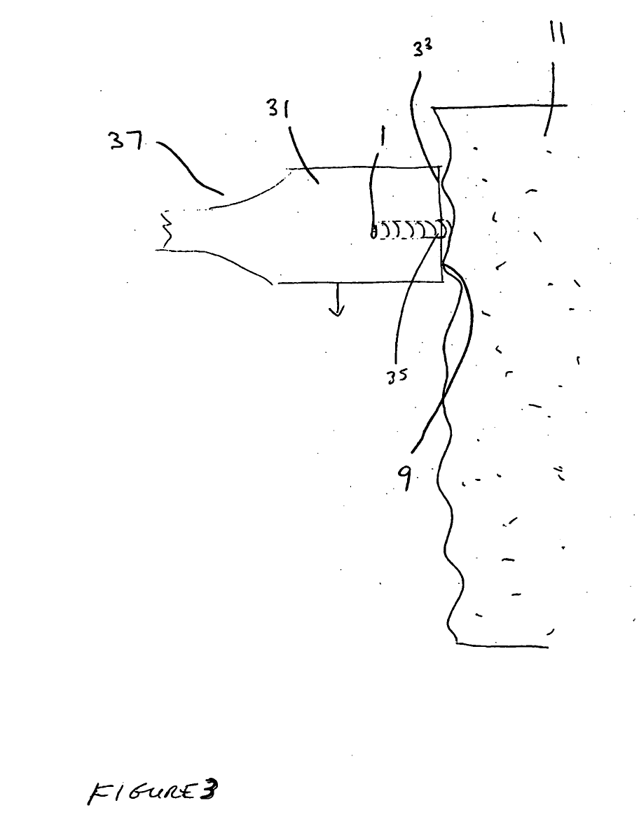

Note that while FIG. 1 depicts the light source 1 as separated from the expanse of skin 11, the distance of separation need not be great. In one embodiment of the invention, as shown in FIG. 3, the light source 1 is a part of a device 37 that includes light source 1 within a housing 31. The housing 31 (e.g., a plastic shell or container) has at least one outer surface, such as a skin-facing surface 33 that may be placed against the outer surface 9 of the expanse of skin 11, such that the light is directed through an optical window 35 to contact the expanse of skin 11. The device may, for example, be held in a user's hand and the incident light 7 may be progressively repositioned across all or portions of the expanse of skin 11. In this embodiment of the invention, the light source 1 may be maintained, for example, a distance of from about 0.5 centimeters (cm) and about 50 cm such as from about 5 cm to about 10 cm from the expanse of skin 11 during operation.

Light Source

The light source 1 suitable for the present invention may provide, for example, a directed beam that is capable of impinging upon the expanse of skin 11 with a relatively small spot size. One suitable light source for generating a narrow spot size is a laser, such as, for example, a semiconductor laser (i.e., a “laser diode”), a ruby laser, or an Nd:YAG laser, an argon laser, a KTP laser, a dye laser, an alexandrite laser or, other lasers that may be capable of emitting light that includes the active region of wavelengths. The laser may emit light in continuous or pulsed fashion. Furthermore, suitable lasers typically have an emitted light 3 with a bandwidth less than about 2 nm. Examples of specific laser light sources that may be used in accordance with the embodiments of the present invention include those described in Altshuler (U.S. Pat. No. 6,273,884) and Anderson (U.S. patent application 20020099094A1), paragraphs 47-49. These disclosures are hereby incorporated by reference.

In another embodiment of the invention, the light source 1 may be a broadband source such as, for example a flashlamp, such as may include an incandescent, fluorescent, or chemiluminescent source. Note that specific examples of particularly suitable light sources are discussed below. Note also that the source 1 may be a broadband source that includes a filament (e.g., a tungsten filament),

Flashlamp

One notable example of a light source that may be used for practicing embodiments of the invention described herein is a pulsed, broadband source is a flashlamp (e.g. a xenon flashlamp). The flashlamp is a gas filled discharge device that takes incident electrical energy, and generates a high voltage electrical pulse that discharges the flashlamp, thereby producing pulses of electromagnetic radiation that fall within a spectral range, such as from about 200 nm to about 2000 nm. The spectral range may be adjusted by selecting a particular fill gas, a particular gas pressure, and a particular current density. Furthermore selection of a particular glass enclosure, or using one or more filters or fluorescent materials may be used to focus the incident energy within a spectral range that is narrower than the spectral range of the emitted electromagnetic radiation.

A flashlamp is suitable for providing benefits to the skin in that it emits emitted light 3 that generally extends widely (in a spatial sense) from the flashlamp, and is therefore capable of simultaneously treating an expanse of skin 11 having a large area. The area over which the light from the flashlamp extends may be limited, however, such as by using reflectors to concentrate the light spatially. The active portion may have a bandwidth that is greater than about 20 nm.

In one embodiment of the invention, the active portion has a bandwidth greater than about 100 nm. The incident light 7 from the flashlamp is generally non-collimated (i.e., the light is emitted in rays that are generally parallel with one another) and non-coherent (the light is emitted in rays that are not phase synchronized with one another). The flashlamp may provide pulses of light that have a duration in a range from about 1 millisecond (msec) to several hundred milliseconds such as from about 10 msec to about 200 msec.

The flashlamp may deliver the particular range of intensity and bandwidth of the active portion that is specified above when the source 1 is placed a distance of, for example, between about 5 cm to about 10 cm (for example, when the outer surface 33 is placed in contact with the surface 9 of the expanse of skin 11).

Incident light 7 of the flashlamp may be high intensity, i.e., the active portion may deliver an energy density that is from about 10 J/cm² to about 100 J/cm². The use of high intensity flashlamp may be may be particularly suitable for use by a skilled user (e.g., a dermatologist, a medical technician, or the like). Alternatively, a high intensity flashlamp may be used for a consumer product if appropriate safety features are employed (e.g., such as those to limit over-treatment to the skin or exposure to the eye). In fact, by having a consumer use a light source having a fluence from about 10 J/cm² to about 100 J/cm², and using methods consistent with embodiments of the invention described herein, the consumer may self-treat with “at home” treatments that are highly efficacious. At home use of such devices allows for more frequent treatments than might be otherwise possible if an appointment to a professional's office were required for each treatment. More frequent treatments, even at lower dose levels provide opportunity for greater compliance and treatment efficacy. A suitable high intensity flashlamp is described in Ekhouse (U.S. Pat. No. 5,405,368), incorporated herein by reference.

Alternatively, the incident light 7 of the flashlamp may be low intensity, i.e., the active portion may have an energy density in a range from about 5 J/cm² to about 10 J/cm². The use of low intensity radiation may be particularly suitable for use by a consumer that may not have any special or professional training in the use of the flashlamp. In general, a suitable low intensity flashlamp will have, for example, a smaller capacitor or a lower voltage than a comparable high intensity flashlamp.

Furthermore, other low intensity sources such as light emitting diodes, filament sources, fluorescent sources, and even chemiluminescent sources can provide skin benefits when used over longer exposure periods (seconds to many minutes) and with more frequent treatments than is typically used in a professional setting.

Light Emitting Diode

Another notable source for practicing embodiments of the present invention is a light emitting diode (LED). The LED is constructed from materials known in the art (e.g., compound semiconductor materials). In one embodiment of the invention, the emitted light 3 from the LED is within (A) about 400 nm to about 500 nm; (B) about 580 nm to about 600 nm; and (C) about 600 nm to about 800 nm. The narrowband source may have an emitted energy density within the active range that is greater than about 0.1 J/cm².

Referring again to FIG. 2B, the emitted light 3 from the LED may be collimated such that it impinges upon the expanse of skin 11 with spot 210 having an area less than about 10 cm². By using a source such as an LED, it is possible to provide an incident energy density that is substantially lower than that of a laser (e.g., laser diode). Radiant intensities of these LEDs may be in the range of about 1 mW/cm² to 10 mW/cm².

As shown in FIG. 3, the LED may be part of a unit such as portable unit having an exposure window across which the light is delivered such that it may contact the expanse of skin 11. The unit, and therefore the light, may be moved along or across the expanse of skin 11 to be treated in order to deliver energy thereto.

The incident light 7 from the narrowband source may be “continuous wave,” (as described in Altschuler U.S. Pat. No. 6,280,473, the disclosure of which is hereby incorporated by reference.). By continuous wave it is meant that the source is adapted to provide a steady-state, uninterrupted beam such that an intensity of the incident light is relatively constant over any time period less than about 1 second.

Note that while the light source 1 is described in this embodiment of the invention as “an LED,” the light source may actually include multiple LEDs in order to enhance the energy density that the light source 1 is capable of delivering.

Benefit Agents and Compositions

Benefit agents of the present invention are generally passive in that they are substantially non-absorptive or otherwise substantially non-interactive with light within the active region. In other words, the benefit agents of the present invention are not necessarily selected in order to absorb incident light from the light source 1 in order to convert the incident light 7 to thermal energy and dissipate the thermal energy to the expanse of skin 11.

In fact, for embodiments of the invention in which the treatment is cyclical (i.e., a second skin treatment is provided following a first skin treatment), it is, to a degree, beneficial that the benefit agent not absorb the incident light 7 to a significant degree. This is because, if benefit agent remains on the expanse of skin 11 when the expanse of skin 11 is treated with light, losses due to absorption by the benefit agent either reduce the ability of the incident light 7 to provide thermal heating to. the expanse of skin 11, and/or require sources of greater power (thus requiring more space, more expense, more cooling of the source, or more expense). In one embodiment of the invention, the benefit agent has an absorbance that is no greater than 0.3 Absorbance Units for any wavelength comprising the incident light 7. This can be determined through spectrophotometric measurements of a thin film of the agent applied to transparent medium, standard in the sunscreen industry, at approximately 2 mg/cm2.

In one embodiment of the invention, the benefit agent is anti-microbial treatment. Examples of suitable anti-microbial treatments include; TRICLOSAN™; methyl, or propyl, paraben, benzyl peroxide, bacitracin, erythromycin, neomycin, tetracycline, chlortetracycline, benzethonium chloride, phenol, sulfur, tricetylmonium chloride, polyquaternium 10, and resorcinol. A particularly noteworthy anti-microbial treatment is benzoyl peroxide.

In one embodiment of the invention, the benefit agent is sebum-modulating treatment. Examples of suitable sebum-modulating treatment treatments include retinoids such as retinol, retinyl palmitate, retinyl propionate, retinaldahyde, retinoic acid, adapelene, tazarotene, 13 cis-retinoic acid, soy extracts; anti-fungals such as miconozole, elubiol, econozole, 5-α-reductase inhibitors, Saw Palmetto Extract, Cedrus Atlantica Bark Extract, Capryloyl Glycine & Sarcosine & Cinnamomum Zeylanicum Bark Extract. Particularly noteworthy sebum-modulating treatments are retinal and retinoic acid.

In another embodiment of the invention, the benefit agent is a keratolytic agent. Examples of suitable keratolytic agents include hydroxyacids such as alpha-hydroxyacids (AHAs), beta-hydroxyacids BHAs, and polyhydroxyacids. Suitable hydroxyacids include: glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucoheptonic acid, glucoheptono 1,4-lactone, gluconic acid, gluconolactone, glucuronic acid, glucuronolactone, glycolic acid, isopropyl pyruvate, methyl pyruvate, mucic acid, pyruvic acid, saccharic acid, saccaric acid 1,4-lactone, tartaric acid, and tartronic acid; beta hydroxy acids such as salicylic acid, beta-hydroxybutyric acid, beta-phenyl-lactic acid, beta-phenylpyruvic acid, azeleic acid; Another useful class of keratolytics are keratolytic enzymes papain, bromaline, pepsin, trypsin.

In one embodiment the benefit agent is an anti-inflammatory agent. Suitable anti-inflammatory agents include: feverfew; alkanolamines such as ethylaminoethanol, methylaminoethanol, dimethylaminoethanolamine (DMAE), isopropanolamine, triethanolamine, isopropanoldimethylamine, ethylethanolamine, 2-butanolamine, choline and serine, catacholamines; hydrocortisone, salicylates, β sitosterol, allantoin, oat extracts, dexamethasone, caffeic acid, ginko bilboa, Stearyl glycyrrhetinate, CM Glucam, green tea extract, hyluronic acid, horsechestnut extract, licorice extract, colloidal oatmeal, tetrahydrozaline, and indomethacin. Alkanolamines such as DMAE, Feverfew, and hydrocortisone are particularly noteworthy anti-inflammatory/anti-redness agents.

In another embodiment of the invention, the benefit agent is scar mitigator such as peptides including Pal-KTTP, Biopeptide EL™, Biopeptide CL™, and copper-containing peptides such as copper polypeptide and copper peptide (GHK). Copper-containing peptides are particularly noteworthy scar mitigators.

In another embodiment of the invention, the benefit agent is an anti-spot/pigmentation agent. Suitable anti-spot/pigmentation agents include: depigmentation agents such as hydroquinone, catechol and its derivatives, ascorbic acid, isoascorbic acid, kojic acid, licorice extract, azelaic acid, stearyl glycyrrhetinate, soy extracts, yohimbine, black tea extracts, and mixtures thereof; kinetin.

In another embodiment of the invention, the benefit agent is a cleansing agent. Suitable cleansing agents include solvents such as lower alcohols including ethanol and isopropanol; and surface active/wetting agents.

The benefit agent may be combined or compounded with various other auxiliary ingredients into a topical personal care composition (e.g., a cream, emulsion, serum, solution, or the like). The selection of the auxiliary ingredients may vary depending upon, for example, the ability of the benefit agent to penetrate through the skin, the specific benefit agent chosen, the particular benefit desired, the sensitivity of the user to the benefit agent, the health condition, age, and skin condition of the user, and the like. Suitable auxiliary agents include fillers, emollients and spreading agents, skin conditioners, emulsifiers, wetting agents, chelating agents, fragrances, thickeners, dyes, sensates, and the like. In one embodiment of the invention, the auxiliary ingredients have a low absorbance with respect to the incident light 7 (such as less than about 0.3 Absorbance Units, as discussed above for the benefit agent).

The benefit agent is used in a “safe and effective amount,” which is an amount that is high enough to deliver a desired skin, hair or nail benefit or to modify a certain condition to be treated, but is low enough to avoid serious side effects, at a reasonable risk to benefit ratio within the scope of sound medical judgment. Unless otherwise expressed herein, typically the benefit agent is present in the personal care composition in an amount, based upon the total weight of the composition/system, from about 0.01 percent to about 20 percent, such as from about 0.01 percent to about 5 percent (e.g., from about 0.01 percent to about 1 percent).

Skin Treatment

In one embodiment of the invention, the expanse of skin 11 to be treated is provided a first skin treatment. The first skin treatment includes exposing the expanse of skin 11 to light primarily within the spectral range of about 400 nm to about 850 nm, said light source delivering from about 5 Joules per square centimeter to about 100 Joules per square centimeter to the skin. The light may be a source of continuous or pulsed light. In the case of pulsed light, terminating a series of pulses terminates the light treatment. After a period that is less than about 1 hour, exposure to the light is terminated. Note that depending upon the fluence of the light, the light may be terminated in a shorter period of time such as within a few minutes, a few seconds or even within less than one second.

Within a first delay period of less than about 24 hours after terminating the exposure to the light, a benefit agent is topically applied. By combining post-treating the expanse of skin 11 with a benefit agent after light treatment within a first delay period of 24 hours or less, a higher order of benefits is provided (i.e., a higher degree of effectiveness and/or a faster onset of benefits is provided as compared with conventional treatments). Without wishing to be bound by theory, it is believed that the inventive treatment regimen operates by multiple biological pathways (e.g., collagen formation and redness reduction). As such, the magnitude or speed of onset of benefits is not limited by the saturation of a single (i.e., light only or topical only) pathway. In order to further enhance the efficacy of the first skin treatment, the first delay may be less than 12 hours, less than 1 hour, such as from about 1 minute to about 1 hour. In particular, it is believed that by reducing the first delay period to such lower levels, a high degree of synergy is obtained between the light treatment and the topical treatment.

After a second delay period, a second skin treatment is optionally provided to the expanse of skin 11. The second skin treatment includes exposing the expanse of skin to light, terminating the exposure of the expanse of skin 11 to the light, followed by topically administering benefit agent. The second skin treatment may, for example, be similar or identical to the first skin treatment. Note that the second delay period is the time elapsed between the application of the benefit agent in the first skin treatment and the initiation of exposure of the expanse of skin 11 to light in the second skin treatment. The second delay period may be of greater duration than the first delay period. Preferably, the second delay period has a greater duration than the first delay period, more preferably a significantly greater duration. Thus, the application of the benefit agent is a post-exposure treatment, not a pre-treatment.

Note that benefit agent may be topically applied one or more times to the expanse of skin 11 during the second delay. The benefit agent topically applied during the second delay may be the same benefit agent or same class of benefit agent applied in the first treatment, or it may be a different benefit agent or a different class of benefit agent. Topical treatments of the benefit agent may be repeated multiple times and on multiple days between light treatments. Topical and light treatments may be administered at home using a handheld light source.

The light and the topically applied benefit agent may be directed to similar benefits (e.g., anti-microbial light followed by an anti-microbial benefit agent; sebum modulating light followed by sebum modulating benefit agent; inflammation-reducing light followed by an anti-inflammatory benefit agent). Because the topical operates through a chemical-biological pathway (the chemistry of the topical directly induces a biological response), and the light operates through an optical-biological pathway (photons of light induce a thermal response, and, in turn, a biological response), the topical and light can act synergistically and achieve a higher order of benefits.

While it is contemplated that the light and the topically applied benefit agent may be directed to similar skin care benefits, this is not required. In one embodiment of the invention the particular topical treatment and particular spectral distribution of light are chosen to complement one another and/or to act on separate, distinct pathways. Examples are provided in the paragraphs below.

For example, the light treatment may have a spectral distribution that is primarily within the spectral range targeted towards anti-microbial action, such as defined by the union of (a) wavelengths between 400 nm and 450 nm; and (b) wavelengths between about 600 nm and about 700 nm. A topical post-treatment complementary to this light treatment may be one or more of: a sebum-modulating agent, a keratolytic agent, an anti-inflammatory agent, a scar mitigator, an anti-pigmentation agent or a cleansing agent.

In another embodiment of the invention, the light treatment may have a spectral distribution that is primarily within the spectral range targeted towards sebum-modulation, such as defined by the union of (c) wavelengths between about 700 nm and 2000 nm; and (d) wavelengths between about 550 nm and 600 nm. The topical post-treatment, complementary to this light treatment may be one or more of: an anti-microbial agent, a keratolytic agent, an anti-inflammatory agent, a scar mitigator, an anti-pigmentation agent or a cleansing agent.

In another embodiment of the invention, the light treatment may have a spectral distribution that is primarily within the spectral range targeted towards inflammation control, such as defined by wavelengths between about 600 nm and about 750 nm. The topical post-treatment, complementary to this light treatment may be one or more of: an anti-microbial agent, a keratolytic agent, a sebum-modulating agent, a scar mitigator, an anti-pigmentation agent or a cleansing agent.

Product and Package

For convenience to the end user, one or more of light sources 1 and one or more benefit agents may be contained within an outer package and sold as a product. The product may further include instructions that indicate to the user that the user should illuminate the skin with the light source 1 and topically apply the benefit agent. The instructions may further indicate that the light source 1 and the benefit agent are to be used together (i.e., applying the benefit agent to the expanse of skin 11 after exposing the expanse of skin 11 to the light source and within about 24 hours), consistent with embodiments of the invention described herein. Note that the product may include a plurality of light sources 1 and/or benefit agents (i.e., one or more light sources 1 and/or one or more benefit agents). These light sources 1 and benefit agents may be, for example, housed in a primary package (e.g., a tube, a jar, a plastic wrap or film, and the like) that is within the outer package.

Embodiments of the invention overcome one or more drawbacks of the prior art by combining the benefits associated with a treatment based on light (i.e., wound repair) with a topical post-treatment that enhances the efficacy of the light treatment. By employing such a therapy subsequent to the light therapy, it is possible to overcome the limitations of the biological response of “light only” or “topical only” therapy by, for example, stimulating a second pathway resulting in faster onset of benefits and a higher magnitude of benefits. By combining moderate fluence of light that primarily within certain range of wavelengths with topical benefit agents, device design flexibility is enhanced since an complex cooling system is not needed, and treatment is highly efficacious as well as safe to use at home. Post treatment application of the topical benefit agents prevents any potential degradation of the active ingredients that may occur during the light exposures, either from direct energy absorption and degradation, or from thermal breakdown from exposures. Since the topicals are not applied before the light exposures, there is virtually unlimited time for absorption into the skin for the benefit delivery and a “wait” time between topical treatment and light exposure as is taught by others is not needed. The following is a description of examples for treating the skin consistent with embodiments of the invention described herein. A person of ordinary skill in the art may perform other methods of the present invention in an analogous manner.

### EXAMPLES

### Example 1

An expanse of skin is treated with a light from a flashlamp light source (such as one having a xenon-filled quartz-envelope and) including any necessary filters to provide a spectral distribution that is primarily within the union of 400 to 450 nm and 600 nm to 700 nm, a bandwidth of 10 nm, a fluence of 5 to 50 J/cm² and delivered in a pulse of less than 1 second, impinges with a spot size of about 5 to 10 cm^{2 on} an expanse of skin. The light source is repositioned (stamped) across adjacent sites to complete treatment over the entire expanse of skin (e.g., an portion of or an entire face).

Within a first time interval of about an hour after the light treatment is completed, a benefit agent comprising salicylic acid is topically applied to the expanse of skin. After about 24 to 48 hours, the above steps (light treatment, then topical treatment after 1 hour) are repeated.

### Example 2

An expanse of skin is treated with a light from flashlamp light source such as one having including any necessary filters to provide a spectral distribution that is primarily within primarily within the union of 400 to 450 nm and 600 nm to 700 nm, a bandwidth of 10 nm, a fluence of 5 to 50 J/cm² and delivered in a pulse of less than 1 second, impinges with a spot size of about 5 to 10 cm on an expanse of skin. The light source is repositioned (stamped) across adjacent sites to complete treatment over the entire expanse of skin.

Within a first time interval of about an hour after the light treatment is completed, a benefit agent comprising an alpha-hydroxy or poly hydroxy acid is topically applied to the expanse of skin. After about 24 to 48 hours, the above steps (light treatment, then topical treatment after 1 hour) are repeated.

### Example 3

An expanse of skin is treated with a light from flashlamp light source such as one having including any necessary filters to provide a spectral distribution that is primarily within primarily within the union of 400 to 450 nm and 600 nm to 700 nm, a bandwidth of 10 nm, a fluence of 5 to 50 J/cm² and delivered in a pulse of less than 1 second, impinges with a spot size of about 5 to 10 cm on an expanse of skin. The light source is repositioned (stamped) across adjacent sites to complete treatment over the entire expanse of skin.

Within a first time interval of about an hour after the light treatment is completed, a benefit agent comprising a retinoid such as retinoic acid is topically applied to the expanse of skin. After about 24 to 48 hours, the above steps (light treatment, then topical treatment after 1 hour) are repeated.

### Example 4

An expanse of skin is treated with a light from flashlamp light source such as one having including any necessary filters to provide a spectral distribution that is primarily within primarily within the union of 400 to 450 nm and 600 nm to 700 nm, a bandwidth of 10 nm, a fluence of 5 to 50 J/cm² and delivered in a pulse of less than 1 second, impinges with a spot size of about 5 to 10 cm on an expanse of skin. The light source is repositioned (stamped) across adjacent sites to complete treatment over the entire expanse of skin.

Within a first time interval of about an hour after the light treatment is completed, a benefit agent comprising benzoyl peroxide or TRICLOSAN is topically applied to the expanse of skin. After about 24 to 48 hours, the above steps (light treatment, then topical treatment after 1 hour) are repeated.

### Example 5

An expanse of skin is treated with a light from flashlamp light source such as one having including any necessary filters to provide a spectral distribution that is primarily within primarily within the union of 400 to 450 nm and 600 nm to 700 nm, a bandwidth of 10 nm, a fluence of 5 to 50 J/cm² and delivered in a pulse of less than 1 second, impinges with a spot size of about 5 to 10 cm on an expanse of skin. The light source is repositioned (stamped) across adjacent sites to complete treatment over the entire expanse of skin.

Within a first time interval of about an hour after the light treatment is completed, a benefit agent comprising an anti-fungal such as elubiol or ketaconazole is topically applied to the expanse of skin. After about 24 to 48 hours, the above steps (light treatment, then topical treatment after 1 hour) are repeated.

### Example 6

An expanse of skin is treated with a light from a flashlamp light source having a spectral distribution of 625 to 700 nm, a bandwidth of 50 nm, a fluence of 5 to 50 J/cm² and delivered in a pulse of less than 1000 seconds, impinges with a spot size of about 400 to 500 cm^{2 on} an expanse of skin (e.g., simultaneously exposing an entire face to light).

Within a first time interval of about an hour after the light treatment is completed, a benefit agent comprising an extract of feverfew or an extract of soy is topically applied to the expanse of skin. After about 24 to 48 hours, the above steps (light treatment, then topical treatment after 1 hour) are repeated.

### Example 7

An expanse of skin is treated with a light from a flashlamp light source having a spectral distribution of 625 to 700 nm, a bandwidth of 50 nm, a fluence of 5 to 50 J/cm² and delivered in a pulse of less than 1000 seconds, impinges with a spot size of about 400 to 500 cm^{2 on} an expanse of skin (e.g., simultaneously exposing an entire face to light).

Within a first time interval of about an hour after the light treatment is completed, a benefit agent comprising DMAE is topically applied to the expanse of skin. After about 24 to 48 hours, the above steps (light treatment, then topical treatment after 1 hour) are repeated.

### Example 8

An expanse of skin is treated with a light from a flashlamp light source having a spectral distribution of 625 to 700 nm, a bandwidth of 50 nm, a fluence of 5 to 50 J/cm² and delivered in a pulse of less than 1000 seconds, impinges with a spot size of about 400 to 500 cm^{2 on} an expanse of skin (e.g., simultaneously exposing an entire face to light).

Within a first time interval of about an hour after the light treatment is completed, a benefit agent comprising benzoyl peroxide or TRICLOSAN is topically applied to the expanse of skin. After about 24 to 48 hours, the above steps (light treatment, then topical treatment after 1 hour) are repeated.

### Example 9

An expanse of skin is treated with a light from a flashlamp light source having a spectral distribution of 575 to 625 nm, a bandwidth of 50 nm, a fluence of 5 to 50 J/cm² and delivered in a pulse of less than 1 second, impinges with a spot size of about 5 to 10 cm^{2 on} an expanse of skin. The light source is repositioned (stamped) across adjacent sites to complete treatment over the entire expanse of skin.

Within a first time interval of about an hour after the light treatment is completed, a benefit agent comprising benzoyl peroxide or TRICLOSAN is topically applied to the expanse of skin. After about 24 to 48 hours, the above steps (light treatment, then topical treatment after 1 hour) are repeated. The preceding method is suitable, for example, to reduce redness present on the expanse of skin.

### Example 10

An expanse of skin is treated with a light from a flashlamp light source having a spectral distribution of 1000 to 1800 nm, a bandwidth of 400 nm, a fluence of 5 to 50 J/cm² and delivered in a time period of less than 1000 seconds, impinges with a spot size of 400 to 500 cm^{2 on} an expanse of skin (e.g., simultaneously exposing an entire face to light).

Within a first time interval of about an hour after the light treatment is completed, a benefit agent comprising an extract of feverfew or soy is topically applied to the expanse of skin. After about 24 to 48 hours, the above steps (light treatment, then topical treatment after 1 hour) are repeated. The preceding method is suitable, for example, to reduce acne scar pigment spots present on the expanse of skin.

### Example 11

An expanse of skin is treated with a light from a flashlamp light source having a spectral distribution of 625 to 700 nm, a bandwidth of 50 nm, a fluence of 5 to 50 J/cm2 and delivered in a pulse of less than 1000 seconds, impinges with a spot size of about 400 to 500 cm2 on an expanse of skin (e.g., simultaneously exposing an entire face to light).

Within a first time interval of about an hour after the light treatment is completed, a benefit agent comprising salicylic acid is topically applied to the expanse of skin. After about 24 to 48 hours, the above steps (light treatment, then topical treatment after 1 hour) are repeated.

### Example 12

An expanse of skin is treated with a light from a flashlamp light source (such as one having a xenon-filled quartz-envelope and) including any necessary filters to provide a spectral distribution that is primarily within the union of 400 to 450 nm and 600 nm to 700 nm, a bandwidth of 10 nm, a fluence of 5 to 50 J/cm² and delivered in a pulse of less than 1 second, impinges with a spot size of about 5 to 10 cm² on an expanse of skin. The light source is repositioned (stamped) across adjacent sites to complete treatment over the entire expanse of skin.

Within a first time interval of about an hour after the light treatment is completed, a benefit agent comprising an anti-inflammatory such as DMAE is topically applied to the expanse of skin. After about 24 to 48 hours, the above steps (light treatment, then topical treatment after 1 hour) are repeated.

### Example 13

An expanse of skin is treated with a light from a flashlamp light source (such as one having a xenon-filled quartz-envelope and) including any necessary filters to provide a spectral distribution that is primarily within the union of 400 to 450 nm and 600 nm to 700 nm, a bandwidth of 10 nm, a fluence of 5 to 50 J/cm² and delivered in a pulse of less than 1 second, impinges with a spot size of about 400 to 500 cm² on an expanse of skin (e.g., simultaneously exposing an entire face to light).

Within a first time interval of about an hour after the light treatment is completed, a benefit agent comprising 10% isopropanol is topically applied to the expanse of skin. After about 24 to 48 hours, the above steps (light treatment, then topical treatment after 1 hour) are repeated.

### Example 14

An expanse of skin is treated with a light from a flashlamp light source having a spectral distribution of 625 to 700 nm, a bandwidth of 50 nm, a fluence of 5 to 50 J/cm² and delivered in a pulse of less than 1000 seconds, impinges with a spot size of about 5 to 10 cm² on an expanse of skin. The light source is repositioned (stamped) across adjacent sites to complete treatment over the entire expanse of skin.

Within a first time interval of about an hour after the light treatment is completed, a benefit agent comprising a copper-containing peptide is topically applied to the expanse of skin. After about 24 to 48 hours, the above steps (light treatment, then topical treatment after 1 hour) are repeated.

While the foregoing is directed to various embodiments of the invention, other and further embodiments may be devised without departing from the basic scope thereof.

What is claimed is:

1. A method of mitigating acne comprising the steps of:
a. exposing an expanse of skin to light;
b. terminating the exposure of the skin to the light; and
c. applying a benefit agent to the expanse of skin after a delay following the termination;
wherein the light is suitable to perform a function selected from the group consisting of exciting porphyrins associated with the expanse of skin into an energetic state suitable for destroying acne-causing micro-organisms, heating lipids present in sebaceous glands within the expanse of skin in order to modulate the flow of sebum in said sebaceous glands, reducing inflammation, and combinations of these functions.

2. A method of mitigating acne comprising the steps of:
a. providing a first skin treatment to an expanse of skin comprising;
i. exposing an expanse of skin to light having a wavelength of about 400 nm to about 850 nm and a fluence of about 5 J/cm² to about 100 J/cm² ;
ii. terminating the exposure of the skin to the light; and
iii. applying a first benefit agent to the expanse of skin after a delay following the termination;
wherein the light is suitable to perform a function selected from the group consisting of exciting porphyrins associated with the expanse of skin into an energetic state suitable for destroying acne-causing micro-organisms, heating lipids present in sebaceous glands within the expanse of skin in order to modulate the flow of sebum in said sebaceous glands, reducing inflammation, and combinations of these functions; and after a delay
b. providing a second skin treatment to the same expanse of skin comprising;.
i. initiating exposure of an expanse of skin to light;
ii. terminating the exposure of the expanse of skin to the light after a period,
iii. and applying the first benefit agent treatment to the expanse of skin after a delay following the termination.

3. A method of promoting a topical composition comprising the step of instructing a user to topically apply said composition to an expanse of skin affected by acne following an exposure of said expanse of skin to light, wherein the light is:
a. substantially free of ultraviolet radiation;
b. has a wavelength primarily of about 400 nm to about 850 nm; and
c. provides a fluence of about 5 J/cm² to about 100 J/cm²;
d. has selected wavelengths and/or wavelength bands, primarily within a wavelength range of about 400 nm to about 850 nm; and
e. delivers from about 0.01 Watt/cm² to about 100 W/cm² to the skin wherein the total fluence delivered is less than 100 J/cm².

4. The method of any of claims 1, 2, and 3, wherein the light exposure terminates after about one hour.

5. The method of any of claims 1, 2, and 3, wherein the benefit agent comprises at least one component that is suitable to provide anti-microbial action that is complementary to either said modulating of said sebum by said band of light, or complementary to said reduction of inflammation by said light.

6. The method of any of claims 1, 2, and 3, wherein the benefit agent comprises at least one component that is suitable to provide sebum-modulating action that is complementary to either said destruction of said acne-causing microorganisms or complementary to said reduction of inflammation by said light

7. The method of any of claims 1, 2, and 3, wherein the benefit agent comprises at least one component that is suitable to provide anti-inflammation that is complementary to either said modulating of said sebum by said band of light, or complementary to said destruction of said acne-causing microorganisms.

8. The method of any of claims 1, 2, and 3, wherein the light has wavelength or band of wavelengths between about 400 nm and about 410 nm.

9. The method of any of claims 1, 2, and 3, wherein the light has wavelength or band of wavelengths between about 630 nm and about 670 nm.

10. The method of claim 1, wherein the light has wavelength or band of wavelengths between about 700 nm and about 1800 nm.

11. The method of any of claims 1, 2, and 3, wherein the light has a bandwidth of less than about 20 nanometers.

12. The method of any of claims 1, 2, and 3, wherein benefit agent is selected from the group consisting of a keratolytic agent, a scar mitigator, an anti-pigmentation agent, a cleansing agent, and combinations of one or more of such agents.

13. The method of claim 12 wherein the scar mitigator comprises at least one peptide.

14. The method of claim 12 wherein the anti-pigmentation agent comprises at least one anti-spot agent.

15. The method of any of claims 1, 2, and 3, wherein benefit agent further comprises an anti-fungal agent.

16. The method of claim 2, wherein the light exposure of the second treatment terminates after about one hour.

17. The method of claim 2, wherein the delay between the first and second skin treatments is greater than the delay between terminating a light treatment and applying the first benefit agent.

18. The method of claim 2, further comprising the step of applying at least one additional benefit agent treatment during the delay between the first and second skin treatments.

19. The method of claim 3, wherein the light exposure is completed within 24 hours prior to said topical application.

20. A kit comprising:
a. a light source that:
i. has a wavelength primarily of about 400 nm to about 800 nm; and
ii. provides a fluence of about 5 J/cm² to about 100 J/cm²;
iii. is suitable to perform a function selected from the group consisting of exciting porphyrins associated with the expanse of skin into an energetic state suitable for destroying acne-causing micro-organisms, heating lipids present in sebaceous glands within the expanse of skin in order to modulate the flow of sebum in said sebaceous glands, reducing inflammation, and combinations of these functions
b. a benefit agent; and
c. instructions directing that at least one treatment of the benefit agent be applied to the skin affected with acne within 24 hours immediately following exposure of skin affected with acne to light from said light source.

21. The kit of claim 20, wherein the benefit agent comprises at least one component that is suitable to provide anti-microbial action that is complementary to either said modulating of said sebum by said band of light, or complementary to said reduction of inflammation by said light.

22. The kit of claim 20, wherein the benefit agent comprises at least one component that is suitable to provide sebum-modulating action that is complementary to either said destruction of said acne-causing microorganisms or complementary to said reduction of inflammation by said light

23. The kit of claim 20, wherein the benefit agent comprises at least one component that is suitable to provide anti-inflammation that is complementary to either said modulating of said sebum by said band of light, or complementary to said destruction of said acne-causing microorganisms.

24. The kit of claim 20, wherein the light has wavelength or band of wavelengths between about 400 nm and about 410 nm.

25. The kit of claim 20, wherein the light has wavelength or band of wavelengths between about 630 nm and about 670 nm.

26. The kit of claim 20, wherein the light has a bandwidth of less than about 20 nanometers.

27. The kit of claim 20, wherein benefit agent is selected from the group consisting of a keratolytic agent, a scar mitigator, an anti-pigmentation agent, a cleansing agent, and combinations of one or more of such agents.

28. The kit of claim 27, wherein the scar mitigator comprises at least one peptide.

29. The kit of claim 27, wherein the anti-pigmentation agent comprises at least one anti-spot agent.

30. The kit of claim 20, wherein benefit agent further comprises an anti-fungal agent.
